(19) United States
(12) Patent Application Publication
(10) Pub. No.: US 2019/0046339 A1
(43) Pub. Date: Feb. 14, 2019

(54) System and Method for Load Dampening for Prosthetic Limbs

(71) Applicant: Ben G. McMilian, Shelton, WA (US)

(72) Inventor: Ben G. McMilian, Shelton, WA (US)

(21) Appl. No.: 15/674,930

(22) Filed: Aug. 11, 2017

(51) Int. Cl. A61F 2/78; A61F 2/66

(52) U.S. Cl. CPC A61F 2/7843; A61F 2002/5016; A61F 2/66

(57) ABSTRACT

Methods and systems for cushioning a residual limb in a prosthetic generally comprise prosthetic limb devices, sockets, bladders, and valve stems. In a preferred embodiment, the residual limb/stump is configured and arranged within the socket and rests on the bladder. The valve stem coupled to the bladder is configured and arranged through the bottom of the socket to allow quick and easy inflation of the bladder. In some embodiments, the present invention also includes upper thigh attachments which may include ball bearings, axles, and thigh straps. In another preferred embodiment, the upper thigh attachment allows the user to securely and adjustably attach the prosthetic limb device to the user's upper thigh.

### BACKGROUND OF THE INVENTION

This invention relates to providing a system for improved comfort and wearability of a prosthetic limb. More particularly, this invention relates to providing a system for load dampening to increase comfort on a residual limb/stump in a prosthetic limb.

### OBJECTS AND FEATURES OF THE INVENTION

A primary object and feature of the present invention is to provide a system overcoming the above mentioned problem.

It is a further object and feature of the present invention to provide such a system to improve the existing state of prosthetic limb devices. The invention improves various aspects of the prosthetic limb including comfort, ease in adjustment of bladder, and particularly ease of adjustment of cushioning under varying loads. The invention further improves the shock absorbing ability of a prosthetic limb.

A further primary object and feature of the present invention is to provide such a system that is efficient, inexpensive, and handy.

Other objects and features of this invention will become apparent with reference to the following descriptions.

### BRIEF SUMMARY OF THE INVENTION

Amputation is a surgical procedure to remove all or part of a limb. An arm, leg, foot, hand, toe, or finger can be amputated. Amputation is performed on the limbs of human bodies in great numbers. Often, amputation is performed due to major trauma, to prevent the spread of cancer when tumors are in the bones or soft tissue, frostbite, and due to congenital limbs. Further, lower limb amputations are performed in the tens of thousands each year due to diabetes alone.

Prosthetics, or artificial body parts, are used to replace amputated limbs. The portion of the limb that is not amputated is referred to as the residual limb or stump. Prosthetics are generally configured to provide some cosmetic benefit and utility in the place of the amputated limb. Prosthetics can vary widely in price, fit, and quality.

Prosthetic limbs can cause discomfort for the wearer. Often, the wearer of the prosthetic finds the limb to cause pain, resulting from chafing, and from the weight load put on the stump at the load bearing point of contact to the prosthetic limb. Accordingly, there is a need for a useful, affordable, comfortable solution for prosthetic limb attachments.

Therefore, the present invention combines unique elements of prosthetic limb attachment with a unique load dampening system consisting of a specially designed wearer adjustable fluid filled bladder.

### BRIEF DESCRIPTION OF THE DRAWINGS

A more complete understanding of the present invention may be derived by referring to the detail description and claims when considered in connection with the following illustrative figures. In the following figures, like reference numbers refer to similar elements and steps throughout the figures.

FIG. 1a shows a perspective view illustrating a prosthetic limb device according to a preferred embodiment of the present invention.

FIG. 1b shows an alternate perspective view illustrating a prosthetic limb device according to a preferred embodiment of the present invention.

FIG. 2 shows a perspective view illustrating the upper components of the prosthetic limb device used to connect the lower components to the wearer's thigh.

FIG. 3 shows a perspective view illustrating the axle mechanism and upper components of the prosthetic limb device.

FIG. 4 shows a cross section illustrating the lower components of the prosthetic limb device.

FIG. 5 shows a cross section view of the socket, bladder, and valve components of the prosthetic limb device.

FIG. 6 shows a perspective view of the bladder used in the prosthetic limb device.

### DETAILED DESCRIPTION OF THE INVENTION

In the following description, and for purposes of explanation, various aspects of the present invention may be described in terms of functional block components and various steps. Such functional blocks may be realized by any number of components configured to perform the specified functions and achieve the various results. In other instances, known structures and devices are shown or discussed more generally in order to avoid obscuring the invention. In many cases, a description of the operation is sufficient to enable one to implement the various forms of the invention. Thus, it should be noted that there are many different and alternative configurations, devices and technologies to which the disclosed inventions may be applied. The full scope of the invention is not limited to the examples that are described below.

By combining a method of adjustment of tension pulling the prosthetic limb device onto the wearer's stump with an adjustable load dampening system, a system of multiple adjustments is provided to the wearer to increase the comfort of wearing a prosthetic limb. The present invention includes both a means to adjust how tightly the socket of the prosthetic limb is pulled onto the stump using a strap and buckle system, as well as a load dampening system using a fluid filled bladder.

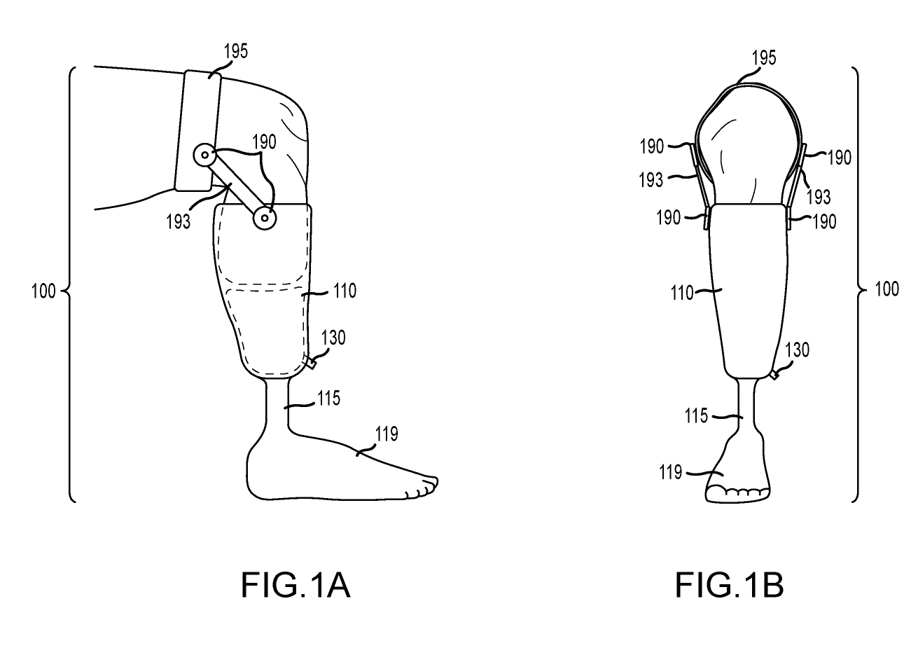

Various representative implementations of the present invention may be applied to any system for artificial limbs. Certain representative implementations may include, for example, an inflatable rubber bladder. Methods and apparatus for cushioning a residual limb in a prosthetic may operate in conjunction with a prosthetic limb device 100. Referring now to FIG. 1(a) and FIG. 1(b), the representation illustrates a prosthetic limb device 100, according to an embodiment. In a non-limiting embodiment, the prosthetic limb device 100 includes a residual limb/stump, a socket, a bladder, and valve stem 130. The stump couples to the socket and the distal end of stump rests on bladder contained within the socket which is configured and arranged within socket 110. The bladder further comprises a valve stem 130 which may be configured and arranged to one side of the central axis through the terminal bottom end of socket 110.

The stump is the residual limb of an amputee. The stump is inserted into the socket and rests on bladder. In the present embodiment, the stump rests within the socket and on bladder. The stump may comprise any amputated limb, such as a residual limb as a result of a below-the-knee amputation, above-the-knee amputation, or an arm amputation. The stump may be further protected within the socket by devices such as a stump sock. In the present embodiment, the stump comprises a residual limb from a below-the-knee amputation.

The socket houses the stump and bladder. In the present embodiment, the socket supports the stump, and bladder is removably configured and arranged within socket. Additionally, valve stem 130 may be configured and arranged through the bottom of socket thus anchoring bladder in socket. The socket may comprise any system for housing the residual limb such as a traditional prosthetic limb which may be intended for a lower leg amputation or an upper leg amputation. In the present embodiment, the socket comprises a lower leg prosthetic.

The bladder protects the stump and maximizes comfort of the wearer. The bladder is configured and arranged within the terminal bottom end of socket where bladder is rested upon by stump and is held in place by valve stem. In the present embodiment, the bladder is coupled to socket by way of valve stem 130.

The bladder may comprise any system for padding the stump, such as an inflatable bladder or a foam pad. In the present embodiment, the bladder comprises an inflatable rubber bladder. For example, the inflatable rubber bladder may comprise a durable PVC bladder and a valve stem 130. The durable PVC bladder is coupled to valve stem 130 at a junction. The valve stem 130 may be configured and arranged through the bottom terminal end of socket. Valve stem 130 may comprise any system for inflating bladder, such a bike pump or an air compressor valve. In the present embodiment, the valve stem 130 comprises a valve that may allow the wearer to adjust the fluid pressure in the bladder while the wearer is wearing the prosthetic limb device 100.

FIG. 1(a) and FIG. 1(b) illustrates an alternative, non-limiting embodiment wherein prosthetic limb device 100 may further comprise upper thigh attachment which itself includes axles 190 and thigh strap 195. In a non-limiting embodiment, the prosthetic limb device 100 includes a residual limb/stump, a socket, a bladder, and valve stem 130 as illustrated in FIG. 1(a) and FIG. 1(b). The stump couples to the socket and the distal end of stump rests on bladder which may be configured and arranged within socket. The bladder may further comprise valve stem 130 which may be configured and arranged through the terminal bottom end of socket.

In addition, axles 190 couple the top end of socket to connecting straps 193. In this embodiment, axles 190 are coupled to socket such that the wearers knee may bend freely. The upper end of the connecting strap is coupled by axles 190 to the thigh strap 195 such that the wearers knee may bend freely.

In the preferred embodiment shown in FIG. 1(a) and FIG. 1(b), the present invention includes prosthetic limb device composed of a socket which contains a fluid filled bladder, a valve stem 130 to allow fluid to be added or removed from the bladder, a lower extension 115, and a support foot 119. Further, the socket is configured and arranged to connect to a thigh strap 195, using rotating axles 190 at each end of connecting straps 193. Alternately preferably, an axle mechanism 190 is used only at the thigh strap 195 end of the connecting strap 193 and the other end of the connecting strap 193 is configured and arranged to be attached to the socket 110 without rotation. Alternately preferably, an axle mechanism 190 is used only at the socket end of the connecting strap 193 and the other end of connecting strap 193 is configured and arranged to be attached to the thigh strap 195 without rotation.

Two leather connecting straps 193 are configured and arranged to extend down the sides of the leg from the support band to the prosthetic socket 110. The dual leather support straps are coupled to the socket of the prosthetic limb device to secure the device to the wearer's upper thigh thus allowing the wearer more security while walking and more comfort while sitting. Further, the addition of the upper thigh attachment allows the wearer to adjust the fit of the prosthetic limb device.

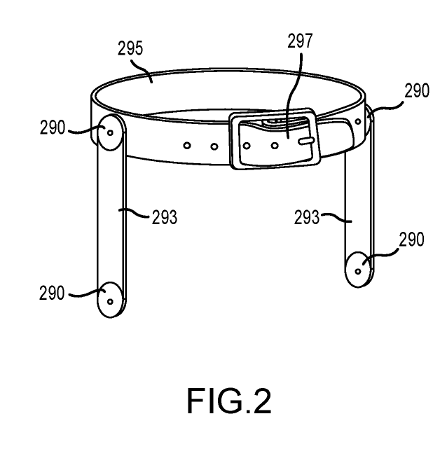

The upper attachment of the prosthetic limb device shown in FIG. 2 includes a thigh strap 295, which is fastened around the wearer's thigh using at least one buckle 297. The buckle 297 allows the wearer to securely fasten the prosthetic limb device to their body, and allows for positioning on the thigh in higher or lower positions so that the tension pulling the socket up against the wearers stump is adjusted, relative to the pressure the stump places on the bladder contained within the socket. Two connecting straps 293 on opposite sides of the limb stump are connected to the thigh strap 295 using an axle 290. The axle 290 is preferably a race bearing, configured and arranged to allow the strap to stay flat while the wearer bends his knee. The socket is also connected to the connecting strap using an axle 290, configured and arranged to allow the connecting strap to remain flat when the wearer bends his knee.

In another preferred embodiment, a plurality of buckles are used on the thigh strap, so that the thigh band may be tightened in a way to allow it to match the couture of the wearer's thigh.

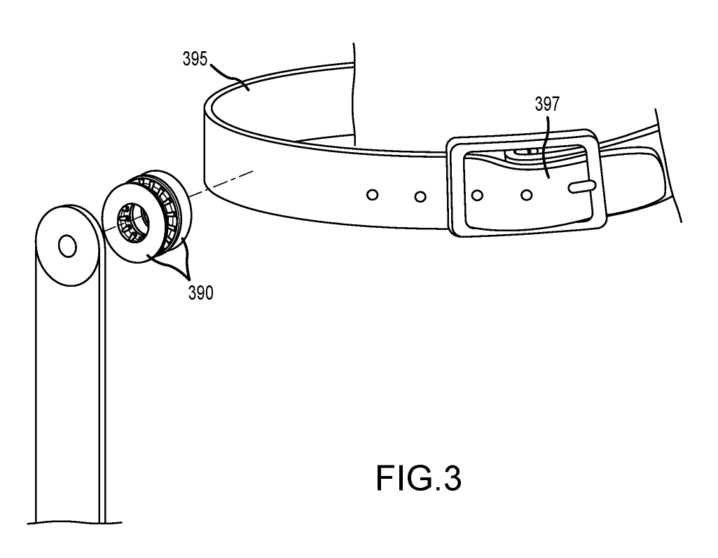

The axle 390 shown in FIG. 3 illustrates how a race bearing can be configured and arranged on the thigh strap 395 allowing rotation along the plane of the connecting strap 393. Alternately preferably, concentric attached rotating axels could be configured and arranged without use of bearings, yet providing rotation along the plane of the connecting strap 393. The thigh strap is fastened around the wearer's thigh using a buckle 397. Alternately preferably, Velcro, latch type, ratchet type, other fasteners could be used in place of a buckle.

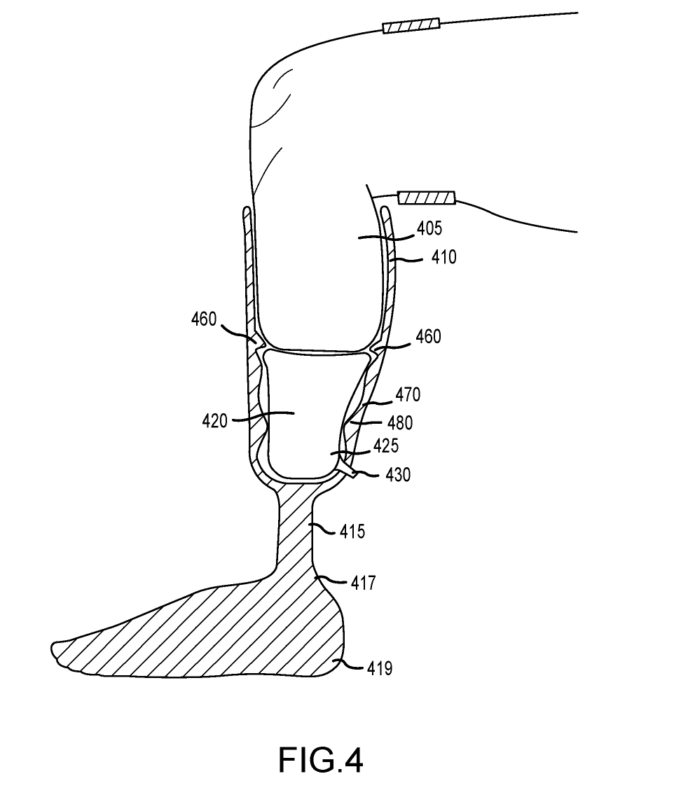

It is shown in FIG. 4 that the wearer's stump 405 is placed into the socket 410, and rests on a flexible fluid filled bladder 420. Alternately preferably, the fluid filled bladder 420 can be filled with air. Alternately preferably, the fluid filled bladder can be filled with nitrogen, argon, or other gasses. Alternately preferably, a compressible non-fluid could be used, such as foam or flexible polymers.

In the preferred embodiment, a valve 430 is configured and arranged on the bladder 420 to pass through the socket 410, so that fluid can easily be added or removed from the external side of the socket, allowing the wearer to adjust the fluid pressure without removing the prosthetic limb system. FIG. 4 further illustrates that a valve 430 serves multiple purposes, including allowing the bladder 420 to be configured and arranged within the stump such that it anchors the position of the bladder in the socket 410, preventing unwanted movements of the bladder while the wearer is wearing the prosthetic limb system.

In addition to the valve 430 providing an anchor point shown in FIG. 4, the bladder is held in place by an upper bulge in the bladder held under an upper ridge 460 in the socket, and a lower bulge 425 in the bladder held in place by a lower ridge 480 in the socket. The bladder 420 is removably placed into the socket such that after inflating the bladder, the upper ridge 460 and lower ridge 480 prevent the bladder from rising out of the bottom of the socket. Alternately preferably, only a lower ridge 480 in the socket, and lower bulge 425 are used without the upper ridge 460 and upper bulge of the bladder 420. Alternately preferably, only an upper ridge 460 and upper bulge of the bladder 420 are used without the lower ridge 480 in the socket and lower bulge 425.

When a load is placed on the bladder 420 by the stump 405, such as when the wearer is standing, the bladder will change in shape. Preferably, the bladder is made of an elastic material, preferably rubber, such that it will expand in size when the stump 405 puts a load on the bladder 420. The bladder 420 is allowed to expand to fill all of the space below the stump 405 to provide a load dampening and cushioning affect for the wearer. Preferably, there is an area specifically provided for expansion, such as the expansion ring 471 as shown in FIG. 4. Alternately preferably, the bladder will expand to any available space below the stump when an expansion ring 471 is not provided.

A lower extension 415 is shown in FIG. 4 that extends below the socket 410 towards the ground, configured and arranged using a flexible component 417 that can simulate the movement of a human ankle, such that it is attached to a foot 419 that comes into contact with the ground.

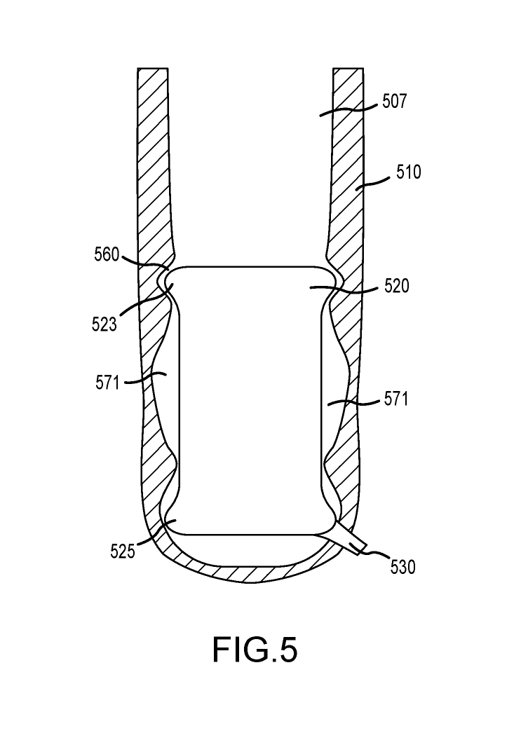

A cutaway of the socket 510, showing the socket opening 507, bladder 520, and valve 530 are shown in FIG. 5. In this preferred embodiment, it is clearly illustrated that the bladder 520 is prevented from rotating in the socket 510 or rising out of the base of the socket. Rotation of the bladder 520 is prevented by anchoring the position of the bladder 520 using the valve 530 which extends through the socket 510 to one side of the center. The bladder 520 is prevented from rising out of the socket 510 by the upper bulge 523 being positioned under the upper ridge 560, and the lower bulge 525 of the bladder 520 being positioned under the lower ridge of the socket.

The expansion area 571 shown in FIG. 5 can be configured and arranged during the manufacturing of the socket 510 such that it is larger or smaller, to aid in the proper tuning of the overall load dampening effects.

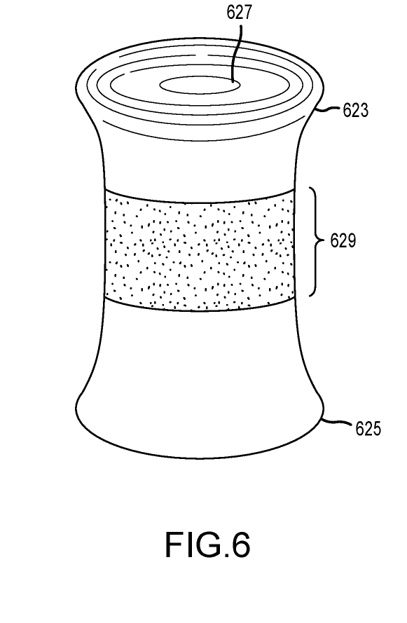

The bladder itself can also be configured and arranged during construction to tune the load dampening affects by using various materials. FIG. 6 illustrates a perspective view of the bladder, having three key regions. The upper bulge 623 of the bladder may be configured and arranged to have a depression 627 on its upper surface, where the depression can be configured and arranged such that it matches the stump of the wearer. In the preferred embodiment, the upper depression is designed by constructing the upper surface to match a three-dimensional map of the wearers stump. Preferably, this is done using laser scanning and mapping of the users stump to acquire information to be used to make a custom matching surface using three-dimensional printing methods. Alternately preferably, one skilled in the art may use heat to shape malleable rubber to the appropriate shape to match the wearers stump. Alternately preferably a casting of the wearer's stump may be made to create a mold of the ideal shape of the upper surface of the bladder. Other methods of shaping an appropriate elastic material will be obvious to one skilled in the art.

The middle section 629 of the bladder shown in FIG. 6 is configured and arranged to be constructed of an elastic material. During the fabrication of the bladder, selection of the elastic material and the material thickness are used to tune the load dampening effect of the bladder. The tuning of load dampening during construction can be widely configured using both this middle section 629, and the size of the expansion area 571 shown in FIG. 5. The lower bulge 625 serves to prevent the bladder from rising above the lower ridge 480 showing in FIG. 4.

In some embodiments, the present invention also includes upper thigh attachments which may include ball bearings, axles, and thigh straps.

In some embodiments, a removable, inflatable bladder is configured and arranged within the socket of a prosthetic limb device and a valve stem to adjust the fluid pressure in the bladder is configured and arranged through the bottom of the socket allowing the wearer to adjust the fluid pressure in the bladder while wearing the prosthetic limb device. This allows the wearer to easily adjust the inflatable bladder. Additionally, the valve stem anchors the inflatable bladder in the prosthetic limb device thus preventing the bladder from moving undesirably. The wearer may adjust the fluid pressure in the bladder via the valve stem to fill the void space in the socket and maximize the comfort of the wearer. As the wearer walks with the prosthetic limb device, the removable inflatable bladder acts as a shock-dampening device minimizing impact on the stump.

In one embodiment, the thigh has a support band around the thigh's circumference which may be adjusted by a buckle. Alternately preferably, a latch mechanism can be used to fasten the support band around the thigh. Alternately preferably, Velcro may be used to fasten the support band around the thigh.

The present invention provides the advantages of being able to be configured and adapted to different load bearing situations. Primarily the present invention is configured and arranged to be comfortable when there is little to no load on the bladder when sitting, and when there is body weight on the bladder when standing. Further, the bladder and support straps can be easily adjusted to accommodate for differing loads, such as if the wearer is carrying heavy items. The present invention provides the advantage of being able to be configured and adapted without removing the prosthetic from the amputated stump.

Although applicant has described applicant's preferred embodiments of this invention, it will be understood that the broadest scope of this invention includes modifications such as diverse shapes, sizes, and materials. Such scope is limited only by the below claims as read in connection with the above specification. Further, many other advantages of applicant's invention will be apparent to those skilled in the art from the above descriptions and the below claims.

1. A prosthetic limb device comprising:
a socket coupled to a stump, wherein said socket comprises a top end and a terminal bottom end; and
an inflatable bladder configured and arranged within said terminal bottom end of said socket such that said stump is cushioned;
wherein said inflatable bladder further comprises a valve stem configured and arranged through an opening in said terminal bottom end of said socket.

2. The prosthetic limb device of claim 1, further comprising an expansion area, configured and arranged within said socket such that said expansion area allows a volume limited expansion of said bladder.

3. The prosthetic limb device of claim 1, wherein said socket further comprising a lower ridge configured and arranged above a lower bulge in said bladder.

4. The prosthetic limb device of claim 1, wherein said inflatable bladder is removable.

5. The prosthetic limb device of claim 1, wherein said valve stem anchors said inflatable bladder to said terminal bottom end of said socket.

6. The prosthetic limb device of claim 1, wherein said inflatable bladder is comprised of a flexible, load bearing material.

7. The prosthetic limb device of claim 1, further comprising an upper thigh attachment, wherein said upper thigh attachment comprising:
at least one ball bearing coupled to said top end of said socket;
at least one axle comprising a first end and a second end, wherein said first end of each axle is coupled to each ball bearing such that said axle rotates freely around said ball bearing; and
at least one thigh strap coupled to said second end of each axle, wherein said at least one thigh strap may removably couple to an upper thigh.

8. The prosthetic limb device of claim 7, further comprising at least one buckle mechanism to fasten said thigh strap to said wearer's thigh.

9. The prosthetic limb device of claim 7, wherein the at least one ball bearings are coupled on opposite sides of a knee joint such that said knee joint bends freely.

10. A method for coupling a prosthetic limb to a stump comprising:
locating said stump within a socket of said prosthetic limb;
coupling said stump to said socket wherein said socket comprises a top end and a terminal bottom end; and
inflating a removable bladder to a comfortable pressure using a valve stem coupled to said bladder, wherein said bladder is configured and arranged within said terminal bottom end of said socket such that said stump is supported and comfortable, to adjust a fluid pressure in said bladder, wherein said valve stem is configured and arranged through an opening in said terminal bottom end of said socket.

11. The method for coupling a prosthetic limb to a stump of claim 10 wherein coupling said prosthetic limb to said stump includes coupling an upper thigh attachment to an upper thigh, wherein said upper thigh attachment comprises:
at least one ball bearing coupled to said top end of said socket;
at least one axle comprising a first end and a second end, wherein said first end of each axle is coupled to each ball bearing such that said axle rotates freely around said ball bearing; and
at least one thigh strap coupled to said second end of each axle, wherein said at least one thigh strap may removably couple to said upper thigh.

12. The method for coupling a prosthetic limb to a stump of claim 10, wherein said pressure of said stump on said bladder is adjusted by a user by positioning of at least one thigh strap.
